(19) United States
(12) Patent Application Publication
(10) Pub. No.: US 2012/0203111 A1
(43) Pub. Date: Aug. 9, 2012

(54) ULTRASONIC DIAGNOSTIC APPARATUS, ULTRASONIC IMAGE PROCESSING APPARATUS, AND ULTRASONIC IMAGE ACQUISITION METHOD

(76) Inventors: Satoshi MATSUNAGA, Nasushiobara-shi (JP); Kazuya Akaki, Utsunomiya-shi (JP); Masaru Ogasawara, Nasushiobara-shi (JP); Yutaka Kobayashi, Nasushiobara-shi (JP); Takayuki Gunji, Otawara-shi (JP)

(21) Appl. No.: 13/365,670

(22) Filed: Feb. 3, 2012

(30) Foreign Application Priority Data
Feb. 4, 2011 (JP) 2011-022999
Feb. 2, 2012 (JP) 2012-020678

(51) Int. Cl. A61B 8/06

(52) U.S. Cl. 600/454

(57) ABSTRACT

According to one embodiment, an ultrasonic diagnostic apparatus includes a detection unit configured to detect a distribution of velocity information at each position in a predetermined area in an object over a predetermined interval by scanning the predetermined area with an ultrasonic wave, a calculation unit configured to calculate at least one feature amount based on at least one of a maximum flow velocity value, a minimum flow velocity value, and a mean flow velocity value at the each position in the predetermined interval by using velocity information at each position over the predetermined interval, and a display unit configured to display the feature amount in a predetermined form.

### CROSS-REFERENCE TO RELATED APPLICATIONS

This application is based upon and claims the benefit of priority from prior Japanese Patent Applications No. 2011-022999, filed Feb. 4, 2011; and No. 2012-020678, filed Feb. 2, 2012, the entire contents of all of which are incorporated herein by reference.

### FIELD

Embodiments described herein relate generally to an ultrasonic diagnostic apparatus, an ultrasonic image processing apparatus, and an ultrasonic image acquisition method.

### BACKGROUND

An ultrasonic diagnostic apparatus emits ultrasonic pulses generated by transducers provided in an ultrasonic probe into an object to be examined, and receives reflected ultrasonic waves generated by differences in acoustic impedance of the tissues of the object via the transducers, thereby acquiring biological information. This apparatus can perform real-time display of image data by the simple operation of bringing the ultrasonic probe into contact with the surface of the body, and hence is widely used for morphological diagnosis and functional diagnosis of various organs.

The above ultrasonic diagnostic apparatus is also used for image diagnosis of the circulatory system. For example, the apparatus measures a blood flow velocity in a specific region at a desired depth from the surface of the body by using a pulse Doppler method, calculates, for example, feature amounts associated with a blood flow such as a PI (Pulsatility Index), RI (Resistance Index), and S/D and flow velocity index values such as a maximum flow velocity value, mean flow velocity value, and minimum flow velocity value, and displays them in real time. The operator can quickly and visually recognize the blood flow state of the patient by observing the displayed blood flow indices.

However, since the conventional apparatus uses the pulse Doppler method for calculating various blood flow indices such as PI, RI, S/D, maximum values, average values, and minimum values, the blood flow indices which the apparatus can calculate are limited to local areas corresponding to one or two rasters. Therefore, the observer (e.g., a doctor) can visually recognize the blood flow in a local area quickly but cannot do so with respect to an area wider than a predetermined area (refer to FIG. 15).

As described above, the blood flow indices which the conventional ultrasonic diagnostic apparatus can calculate are limited to those in a local area. Therefore, if the conventional ultrasonic diagnostic apparatus is used for measuring the blood flow velocity in the entire cervical vessel, a target blood vessel has to be first visualized in a long axis view and then the entire blood vessel has to be visually observed from one portion to another to detect an abnormality, while simultaneously moving the pulse-Doppler sampling position (the gate position) along the long axis of the blood vessel. This being so, a physical burden is imposed on a patient, and an operation burden is imposed on a doctor.

Under the above circumstances, the object is to provide an ultrasonic diagnostic apparatus, an ultrasonic image processing apparatus and an ultrasonic image processing method, which enable calculation of a blood-vessel feature amount for a wider area than before and which enable the observer to visually recognize the calculation result quickly and easily.

### BRIEF DESCRIPTION OF THE DRAWINGS

FIG. 1 is a block diagram showing the arrangement of an ultrasonic diagnostic apparatus 1 according to an embodiment;

FIG. 2 is a flowchart showing a procedure for processing (wide-area feature amount image generation processing) based on a wide-area feature amount generation function;

FIG. 3 is a view showing an example of color raw data for each frame which is generated by a blood flow detection unit 24 and stored in a raw data memory 25;

FIG. 4 is a view showing an example of information stored in a flow index value storage unit 260 and a feature amount storage unit 261 in blood flow information/feature amount information calculation processing;

FIG. 5 is a view showing an example of a wide-area feature amount image displayed while being superimposed on a B-mode image on a monitor 14;

FIG. 6 is a view for explaining the wide-area feature amount image generation function according to the first modification;

FIG. 7 is a view for explaining the wide-area feature amount image generation function according to the second modification;

FIG. 8 is a view for explaining the wide-area feature amount image generation function according to the third modification;

FIG. 9 is a view for explaining the wide-area feature amount image generation function according to the fourth modification;

FIG. 10 is a view for explaining the wide-area feature amount image generation function according to the fifth modification;

FIG. 11 is a view for explaining the wide-area feature amount image generation function according to the sixth modification;

FIG. 12 is a view for explaining the wide-area feature amount image generation function according to the sixth modification;

FIG. 13 is a view for explaining the wide-area feature amount image generation function according to the seventh modification;

FIG. 14 is a view for explaining the wide-area feature amount image generation function according to the seventh modification; and

FIG. 15 is a view for explaining a conventional pulse Doppler method.

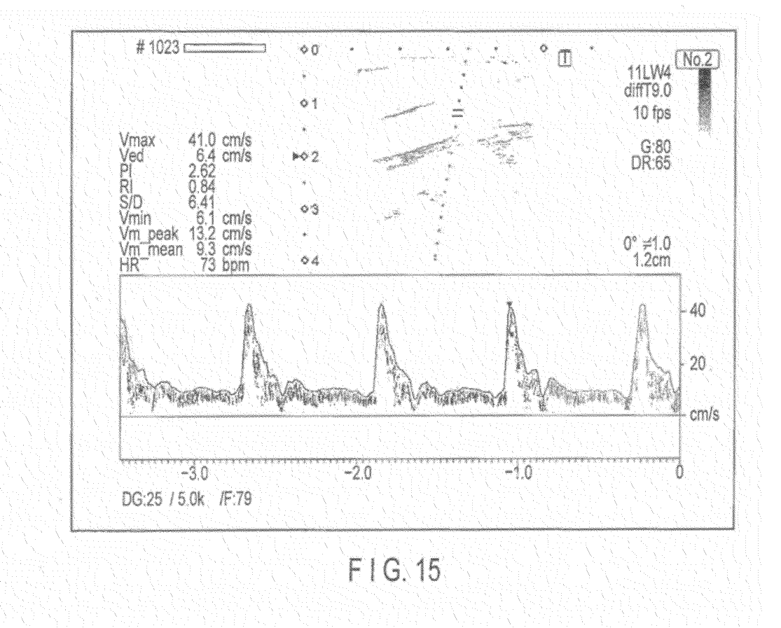

### DETAILED DESCRIPTION

In general, according to one embodiment, an ultrasonic diagnostic apparatus includes a detection unit configured to detect a distribution of velocity information at each position in a predetermined area in an object over a predetermined interval by scanning the predetermined area with an ultrasonic wave, a calculation unit configured to calculate at least one feature amount based on at least one of a maximum flow velocity value, a minimum flow velocity value, and a mean flow velocity value at the each position in the predetermined interval by using velocity information at each position over the predetermined interval, and a display unit configured to display the feature amount in a predetermined form.

The embodiment will be described below with reference to the accompanying drawings. Note that the same reference numerals in the following description denote constituent elements having almost the same functions and arrangements, and a repetitive description will be made only when required.

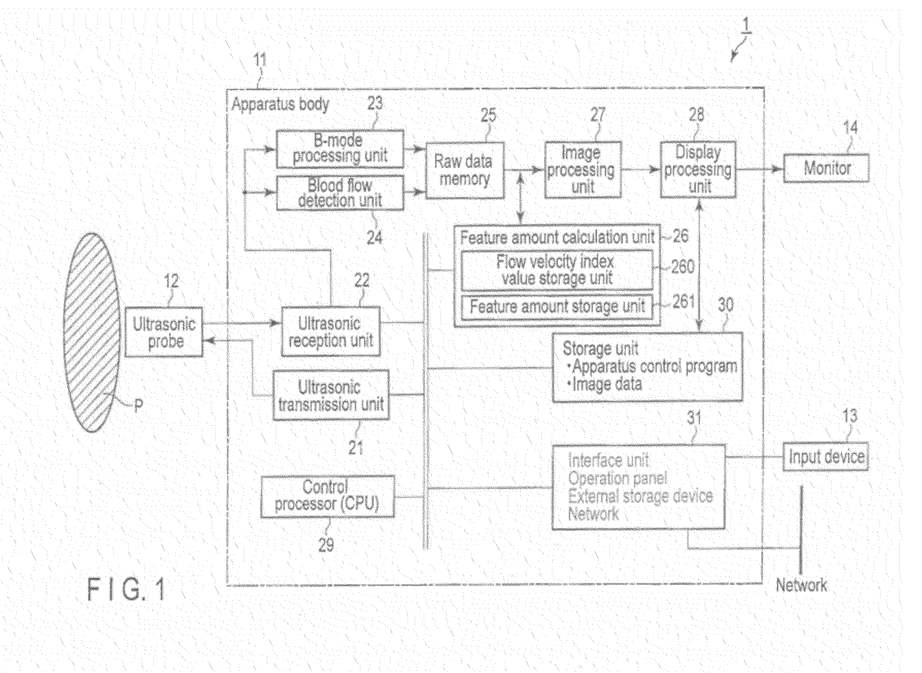

FIG. 1 is a block diagram showing the arrangement of an ultrasonic diagnostic apparatus 1 according to this embodiment. As shown in FIG. 1, the ultrasonic diagnostic apparatus 1 includes an ultrasonic probe 12, an input device 13, a monitor 14, an ultrasonic transmission unit 21, an ultrasonic reception unit 22, a B-mode processing unit 23, a blood flow detection unit 24, a raw data memory 25, a feature amount calculation unit 26, an image processing unit 27, a display processing unit 28, a control processor (CPU) 29, a storage unit 30, and an interface unit 31. The function of each constituent element will be described below.

The ultrasonic probe 12 is a device (probe) which transmits ultrasonic waves to an object, and receives reflected waves from the object based on the transmitted ultrasonic waves. The ultrasonic probe 12 has, on its distal end, an array of a plurality of piezoelectric transducers, a matching layer, a backing member, and the like. The piezoelectric transducers transmit ultrasonic waves in a desired direction in a scan area based on driving signals from the ultrasonic transmission unit 21, and convert reflected waves from the object into electrical signals. The matching layer is an intermediate layer which is provided for the piezoelectric transducers to make ultrasonic energy efficiently propagate. The backing member prevents ultrasonic waves from propagating backward from the piezoelectric transducers. When the ultrasonic probe 12 transmits an ultrasonic wave to an object P, the transmitted ultrasonic wave is sequentially reflected by the discontinuity surface of an acoustic impedance of an internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by a moving blood flow is subjected to a frequency shift depending on the velocity component of the moving body in the ultrasonic transmission/reception direction due to the Doppler effect.

Note that the ultrasonic probe 12 according to this embodiment may be a two-dimensional array probe (i.e., a probe having ultrasonic transducers arranged in the form of a two-dimensional matrix) or a probe which can acquire volume data, e.g., a mechanical 4D probe (i.e., a probe which can execute ultrasonic scanning while mechanically swinging an ultrasonic transducer array in a direction perpendicular to the array direction). Obviously, the ultrasonic probe 12 may be a one-dimensional array probe.

The input device 13 is connected to an apparatus body 11 and includes various types of switches, buttons, a trackball, a mouse, and a keyboard which are used to input, to the apparatus body 11, various types of instructions, conditions, an instruction to set a region of interest (ROI), various types of image quality condition setting instructions, and the like from an operator.

The monitor 14 displays morphological information and blood flow information in the living body as images based on video signals from the display processing unit 30.

The ultrasonic transmission unit 21 includes a trigger generation circuit, delay circuit, and pulser circuit (none of which are shown). The trigger generation circuit repetitively generates trigger pulses for the formation of transmission ultrasonic waves at a predetermined rate frequency fr Hz (period: 1/fr sec). The delay circuit gives each trigger pulse a delay time necessary to focus an ultrasonic wave into a beam and determine transmission directivity for each channel. The pulser circuit applies a driving pulse to the probe 12 at the timing based on this trigger pulse.

The ultrasonic transmission unit 21 has a function of instantly changing a transmission frequency, transmission driving voltage, or the like to execute a predetermined scan sequence in accordance with an instruction from the control processor 29. In particular, the function of changing a transmission driving voltage is implemented by linear amplifier type transmission circuit capable of instantly switching its value or a mechanism of electrically switching a plurality of power supply units.

The ultrasonic reception unit 22 includes an amplifier circuit, A/D converter, delay circuit, and adder (none of which are shown). The amplifier circuit amplifies an echo signal received via the probe 12 for each channel. The A/D converter converts each analog echo signal into a digital echo signal. The delay circuit gives the digitally converted echo signals delay times necessary to determine reception directivities and perform reception dynamic focusing.

The adder then performs addition processing for the signals. With this addition, a reflection component from a direction corresponding to the reception directivity of the echo signal is enhanced to form a composite beam for ultrasonic transmission/reception in accordance with reception directivity and transmission directivity.

The B-mode processing unit 23 receives an echo signal from the reception unit 22, and performs logarithmic amplification, envelope detection processing, and the like for the signal to generate data whose signal intensity is expressed by a luminance level.

The blood flow detection unit 24 extracts a blood flow signal from the echo signal received from the ultrasonic reception unit 22, and generates blood flow data. In general, the blood flow detection unit 24 extracts a blood flow by CFM (Color Flow Mapping). In this case, the blood flow detection unit 24 analyzes the blood flow signal to obtain blood flow information such as mean velocities, variances, and powers as blood flow data at multiple points.

The raw data memory 25 generates B-mode raw data as B-mode data on ultrasonic scanning lines by using a plurality of B-mode data received from the B-mode processing unit 23. The raw data memory 25 generates color raw data as blood flow data on ultrasonic scanning lines by using a plurality of blood flow data received from the blood flow detection unit 24. Note that for the purpose of reducing noise or smooth concatenation of images, a filter may be inserted after the raw data memory 25 to perform spatial smoothing.

With the wide-area feature amount image generation function (to be described later), the feature amount calculation unit 26 receives blood flow information obtained by CFM over a predetermined interval from the raw data memory 25, and calculates a flow index value and feature amount associated with a blood flow at each position in the blood vessel under the control of the control processor 29. In this case, a flow index value associated with a blood flow is, for example, a maximum value, mean value, or minimum value, and a feature amount associated with a blood flow is, for example, a PI, RI, or S/D.

The image processing unit 27 generates B-mode image data, CFM image data, and volume data by using the B-mode raw data and color raw data received from the raw data memory 25. The image processing unit 27 performs predetermined image processing such as volume rendering, MPR (Multi Planar Reconstruction), and MIP (Maximum Intensity Projection). The image processing unit 27 generates a feature amount image in which different colors are assigned in accordance with feature amount values by using the feature amounts at the respective positions which are calculated by the feature amount calculation unit 26.

Note that for the purpose of reducing noise or smooth concatenation of images, a two-dimensional filter may be inserted after the image processing unit 27 to perform spatial smoothing.

The display processing unit 28 executes various kinds of processes associated with a dynamic range, luminance (brightness), contrast, y curve correction, RGB conversion, and the like for various kinds of image data generated/processed by the image processing unit 27.

The control processor 29 has the function of an information processing apparatus (computer) and controls the operation of the main body of this ultrasonic diagnostic apparatus. The control processor 29 reads out a dedicated program for implementing a wide-area feature amount image generation function (to be described later) from the storage unit 30, and the like from a storage unit 30, expands the program in its own memory, and executes computation, control, and the like associated with each type of processing.

The storage unit 30 stores the dedicated program for implementing the wide-area feature amount image generation function, diagnosis information (patient ID, findings by doctors, and the like), a diagnostic protocol, transmission/reception conditions, a color table for assigning different colors in accordance with calculated feature amount values, and other data groups. The storage unit 30 is also used to store images in the image memory (not shown), as needed. It is possible to transfer data in the storage unit 30 to an external peripheral device via the interface unit 31.

The interface unit 31 is an interface associated with the input device 13, a network, and a new external storage device (not shown). The interface unit 31 can transfer, via a network, data such as ultrasonic images, analysis results, and the like obtained by this apparatus to another apparatus.

### (Wide-Area Feature Amount Image Generation Function)

The wide-area feature amount image generation function of the ultrasonic diagnostic apparatus 1 will be described next. This function serves to generate a feature amount image such that flow velocity index values and feature amounts associated with blood flows at the respective positions in the blood vessel are calculated using blood flow information obtained by CFM over a predetermined interval and different colors are assigned in accordance with the feature amount values. This makes it possible to quickly and easily observe feature amounts in a wide area as compared with the prior art.

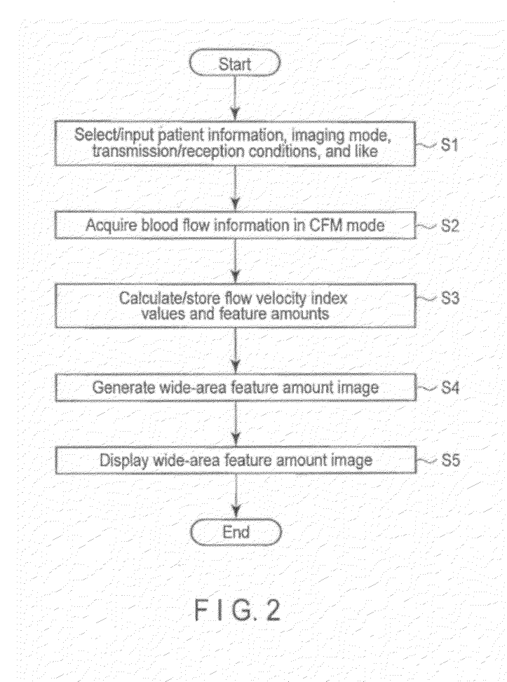

FIG. 2 is a flowchart showing a procedure for processing (wide-area feature amount image generation processing) based on the wide-area feature amount image generation function. The contents of processing in each step will be described below.

### [Reception of Input of Patient Information and Transmission/Reception Conditions: Step S1]

The operator executes, via the input device 13, inputting of patient information and selection of an imaging mode, scan sequence, transmission/reception conditions, and the like for ultrasonically scanning a predetermined area in an object (step S1). In this case, the operator selects the CFM mode as an imaging mode, and inputs a sample volume, transmission voltage, and the like as transmission/reception conditions. The storage unit 30 automatically stores the input and selected pieces of information, conditions, and the like.

### [Acquisition of Blood Flow Information in CFM Mode: Step S2]

The operator brings the ultrasonic probe 12 into contact with the surface of the object at a desired position to execute ultrasonic scanning in the CFM mode for an area including a diagnosis region (a desired blood vessel in this case) as an area to be scanned. The echo signals acquired by ultrasonic scanning in the CFM mode are sent to the blood flow detection unit 24 via the ultrasonic reception unit 22. The blood flow detection unit 24 extracts blood flow signals by CFM, obtains blood flow information such as mean velocities, variances, and powers as blood flow data at multiple points, and generates blood flow velocity information (color data) for each frame. The raw data memory 25 generates color raw data for each frame by using a plurality of color data received from the blood flow detection unit 24 (step S2).

### [Calculation of Flow Velocity Index Value and Feature Amount: Step S3]

The feature amount calculation unit 26 receives blood flow information, of the blood flow information obtained by CFM, which corresponds to a predetermined interval from the raw data memory 25, and calculates a flow velocity index value and a feature amount at each position in the blood vessel (step S3).

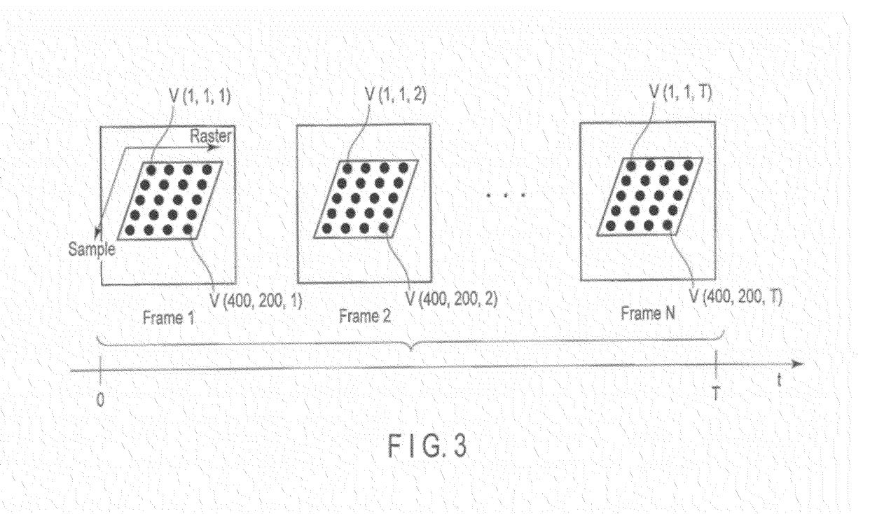

FIG. 3 is a view showing an example of the color raw data for each frame which is generated by the blood flow detection unit 24 and stored in the raw data memory 25. FIG. 3 shows an example in which velocity information corresponding to a sample x and raster y in the nth frame, generated by the blood flow detection unit 24, is defined as V(x, y, n), and a frame count, sample count, and raster count are respectively set to N, 400, and 200. In addition, the maximum velocity, minimum velocity, and mean velocity at the sample x and the raster y in the range from the first frame to the

Nth frame are respectively defined as Vmax(x, y), Vmin(x, y), and Vmean(x, y). Furthermore, PI(x, y) and RI(x, y) at the sample x and the raster y in the range from the first frame to the Nth frame are respectively defined by equations (1) and (2) given below:

PI(x,y)=(Vmax(x,y)−Vmin(x,y))/Vmean(x,y) (1)

RI(x,y)=(Vmax(x,y)−Vmin(x,y))/Vmax(x,y) (2)

Upon receiving velocity information V(x, y, 1) (where x and y are natural numbers satisfying 1≦x≦400 and 1≦y≦200) of the first frame from the raw data memory 25, the feature amount calculation unit 26 temporarily stores the information in its memory.

Upon receiving velocity information V(x, y, 2) of the second frame from the raw data memory 25, the feature amount calculation unit 26 temporarily stores the information in its memory, and compares the information with the velocity information V(x, y, 1) of the first frame to calculate the maximum velocity Vmax(x, y), minimum velocity Vmin(x, y), and mean velocity Vmean(x, y) at the sample x and the raster y. In addition, the feature amount calculation unit 26 calculates PI(x, y) and RI(x, y) by using obtained Vmax(x, y), Vmin(x, y), and Vmean(x, y) according to equations (1) and (2). The feature amount calculation unit 26 stores acquired Vmax(x, y), Vmin(x, y), and Vmean(x, y) in a flow velocity index value storage unit 260, and also stores PI(x, y) and RI(x, y) in a feature amount storage unit 261.

Upon receiving velocity information V(x, y, 3) of the third frame from the raw data memory 25, the feature amount calculation unit 26 temporarily stores the information in its memory, and compares the velocity information V(x, y, 3) with the maximum velocity Vmax(x, y) of the first and second frames. If the velocity information V(x, y, 3) is larger than the maximum velocity Vmax(x, y) of the first and second frames, the feature amount calculation unit 26 updates the maximum velocity Vmax(x, y). If the velocity information V(x, y, 3) is smaller than the maximum velocity Vmax(x, y) of the first and second frames, the feature amount calculation unit 26 keeps the maximum velocity Vmax(x, y). The feature amount calculation unit 26 calculates Vmin(x, y) in the same manner, and calculates the average velocity Vmean(x, y) of the first to third frames by using the pieces of velocity information of the first, second, and third frames (or the average velocity Vmean(x, y) of the first and second frames or the velocity information V(x, y, 3) of the third frame). The feature amount calculation unit 26 also calculate PI(x, y) and RI(x, y) by using obtained Vmax(x, y), Vmin(x, y), and Vmean(x, y) according to equations (1) and (2). The feature amount calculation unit 26 then stores acquired Vmax(x, y), Vmin(x, y), and Vmean(x, y) in the flow velocity index value storage unit 260, and PI(x, y) and RI(x, y) in the feature amount storage unit 261.

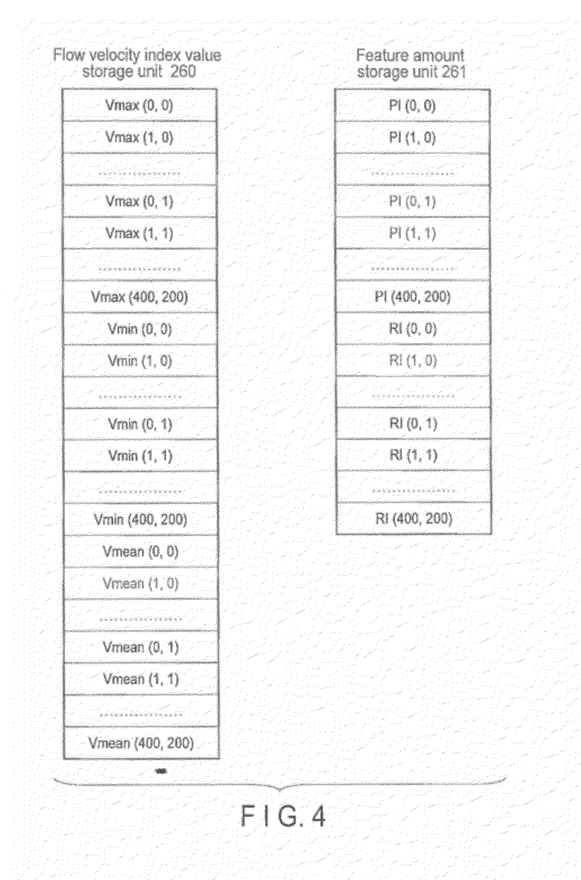

Thereafter, the feature amount calculation unit 26 sequentially executes similar processing up to the Nth frame. As a result, the flow velocity index value storage unit 260 and the feature amount storage unit 261 store pieces of information like those shown in FIG. 4.

Note that it is not necessary to calculate PI(x, y) and RI(x, y) and store (update) them in the feature amount storage unit 261 at the same timings as those for the calculation of Vmax(x, y), Vmin(x, y), and Vmean(x, y) and storing (updating) of them in the flow velocity index value storage unit 260. For example, the apparatus may detect heartbeats based on biological information such as blood flow information and ECG information obtained by CFM, calculate PI(x, y) and RI(x, y) with reference to them (e.g., for each heartbeat, and store (update) them in the feature amount storage unit 261.

### [Generation/Display of Wide-Area Feature Amount Image: Steps S4 and S5]

The image processing unit 27 then generates a wide-area feature amount image by using the acquired blood flow information (step S4). That is, the image processing unit 27 generates a wide-area feature amount image with each feature amount being represented by PI(x, y) by assigning different colors in accordance with the obtained values of PI(x, y) in step S4. The image processing unit 27 also generates a wide-area feature amount image with each feature amount being represented by RI(x, y) by assigning different colors in accordance with the values of RI(x, y) obtained in step S4. The monitor 14 displays the generated wide-area feature amount images in a predetermined form after predetermined display processing is performed on the image (step S5).

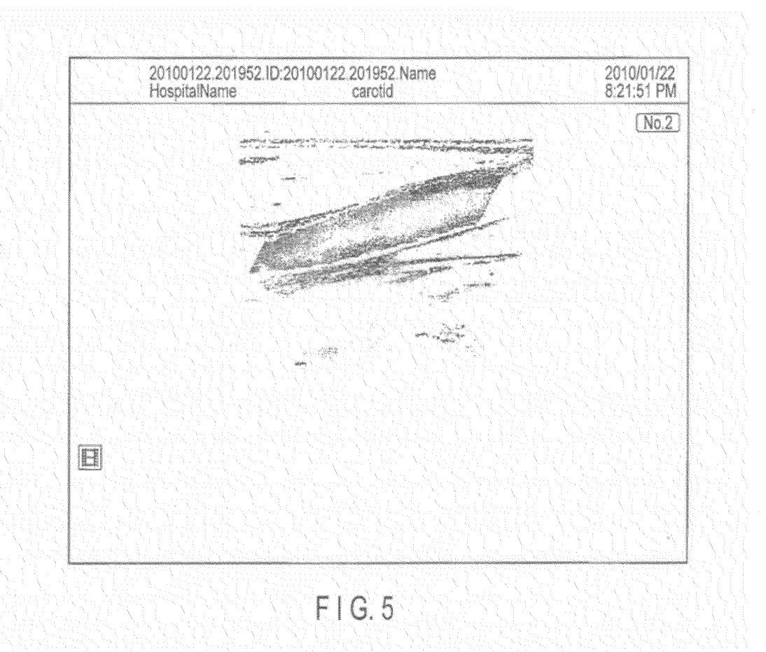

FIG. 5 is a view showing an example of the wide-area feature amount image displayed while being superimposed on a B-mode image on the monitor 14. As shown in FIG. 5, it is possible to visualize PI(x, y) or RI(x, y) in an area winder than that in the prior art.

The above wide-area feature amount image generation function can be variously modified. Typical modifications of this wide-area feature amount image generation function will be described below.

### First Modification

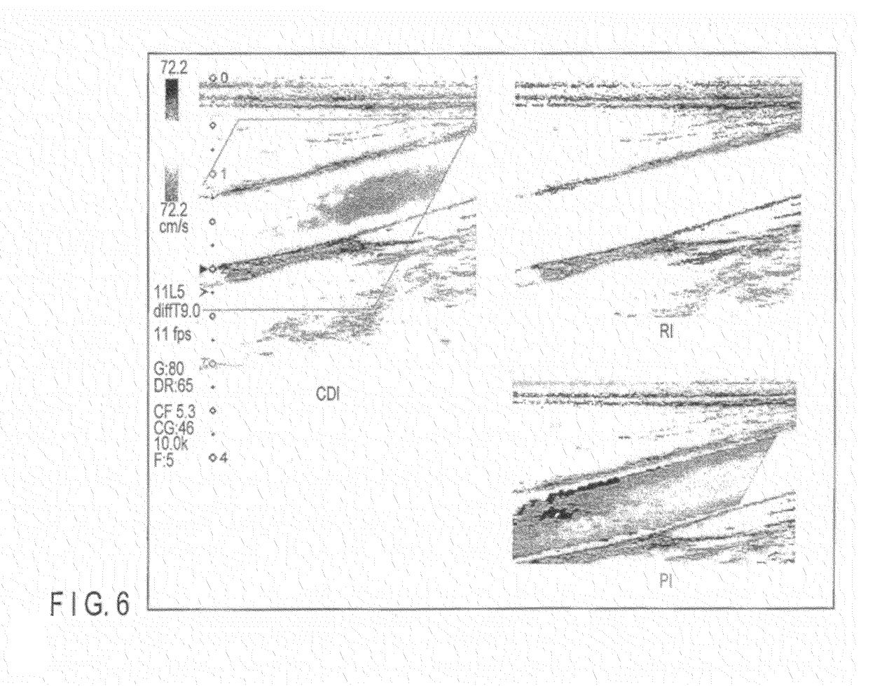

As shown in FIG. 6, the wide-area feature amount image generation function according to the first modification displays a CDI image and a wide-area feature amount image side by side. According to this modification, the observer can visually recognize a blood flow velocity and a blood flow feature amount quickly and easily by simultaneously observing the CDI image and wide-area feature amount image which are simultaneously displayed. In particular, the wide-area feature amount image uses velocity information acquired by CFM, and hence the CDI and the wide-area feature amount image in a wide range are displayed almost simultaneously. This makes it possible to easily associate or compare the CDI image with the wide-area feature amount image, thus improving the observation efficiency.

### Second Modification

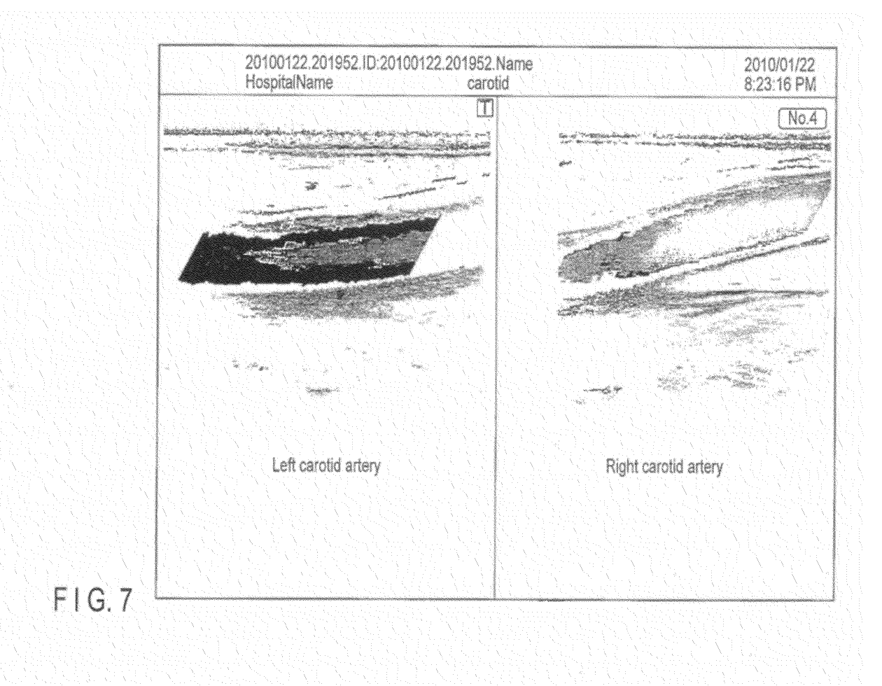

The wide-area feature amount image generation function according to the second modification simultaneously displays wide-area feature amount images of a plurality of slices, as shown in FIG. 7, when, for example, the observer wants to compare wide-area feature amount images of the left and right carotid arteries. This modification allows the observer to quickly and easily compare the feature amounts of spatially separate regions by observing a plurality of wide-area feature amount images which are simultaneously displayed.

Note that the display form according to the second modification is effective, for example, when the operator wants to simultaneously observe a plurality of wide-area feature amount images acquired at different timings.

### Third Modification

The wide-area feature amount image generation function according to the third modification spatially associates a plurality of wide-area feature amount images to display them as one composite image (also called a fusion image, concatenated image, combined image, or panorama image). It is possible to generate this composite image by, for example, calculating moving amounts from changes in image between B-mode frames and concatenating a plurality of wide-area feature amount images upon spatially associating them with each other.

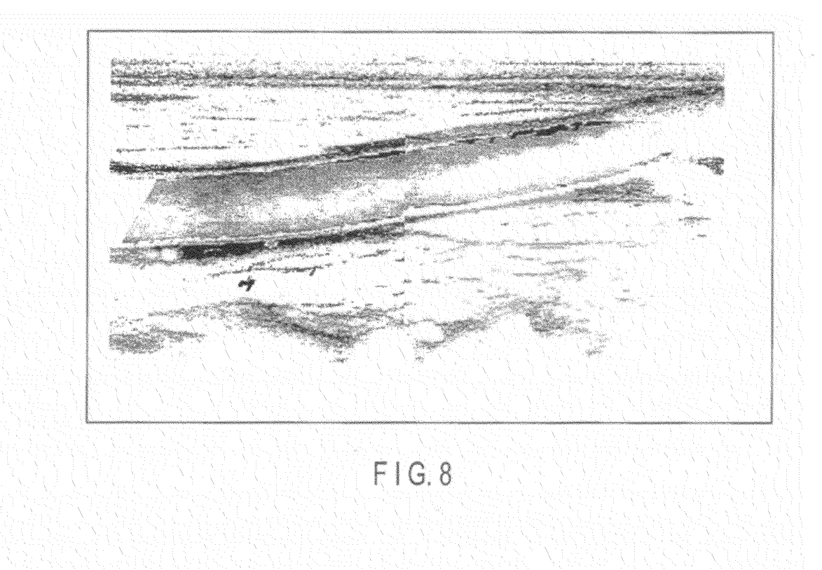

FIG. 8 shows an example of the composite image according to the third modification generated from a plurality of wide-area feature amount images. As is obvious from the comparison between FIGS. 8 and 5, the composite image allows quick and easy visual recognition of the feature amounts of blood flows in a wider area.

### Fourth Embodiment

The wide-area feature amount image generation function according to the fourth modification displays calculated feature amount information and a calculated flow velocity index value as character information.

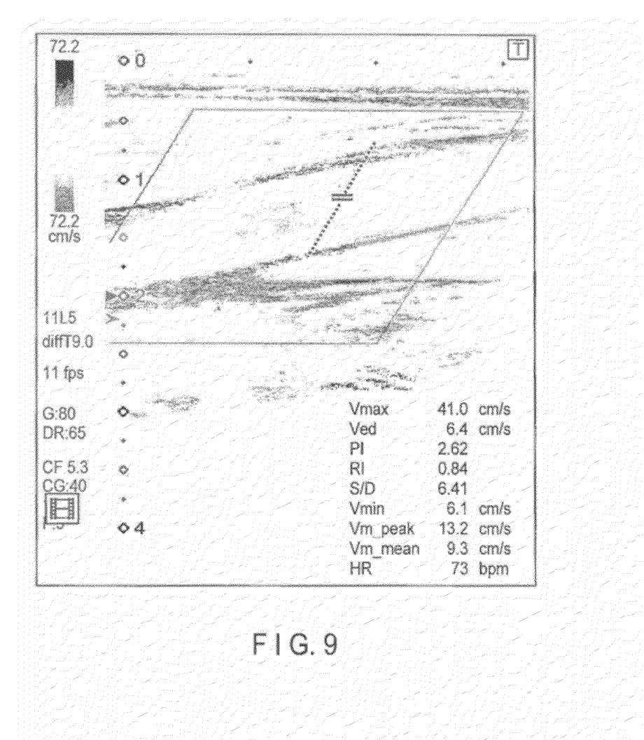

FIG. 9 is a view for explaining a display form according to the fourth modification. As shown in FIG. 9, displaying feature amount information such as a PI and blood flow information such as Vmax as character information together with, for example, a CDI image (or a predetermined wide-area feature amount image) also allows quick and easy visual recognition of the flow velocity index value and the blood flow feature amount.

### Fifth Modification

The wide-area feature amount image generation function according to the fifth modification displays calculated feature amount information in the form of a graph.

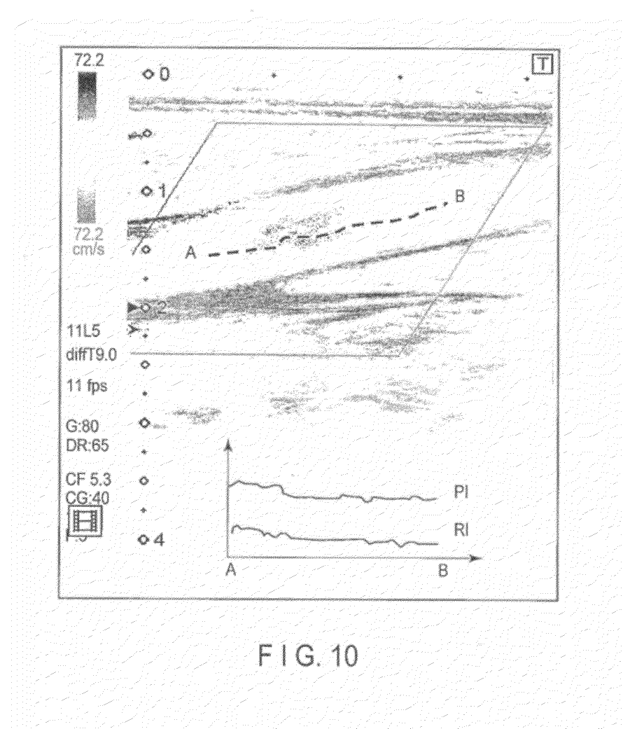

FIG. 10 is a view for explaining a display form according to the fifth modification. As shown in FIG. 10, for example, setting a desired path A-B on a CDI image via the input device 13 will generate a graph showing the spatial change rates of PI and RI along the path A-B based on calculation results. This graph is displayed as shown in FIG. 10. Displaying feature amount information in the form of a graph in this manner also allows quick and easy visual recognition of flow velocity index values and blood flow feature amounts.

### Sixth Modification

The wide-area feature amount image generation function according to the sixth modification specifies a desired position (e.g., a position where the PI value is large) on a wide-area feature amount image, and executes pulse Doppler processing upon automatically setting a sampling position at the specified position.

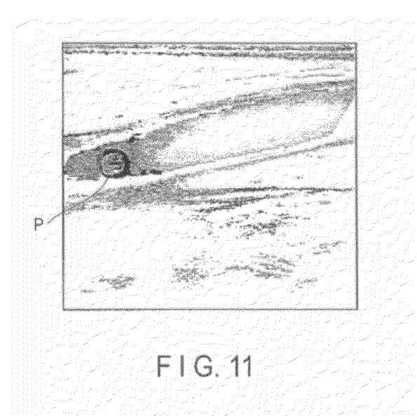

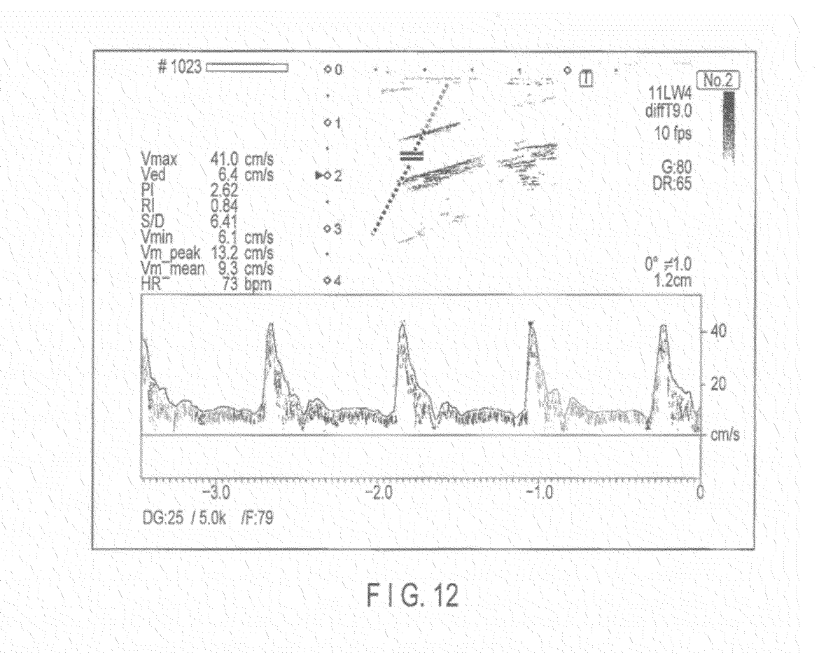

Assume that a wide-area feature amount image like that shown in FIG. 11 is to be acquired and displayed. In this case, when the operator inputs an instruction to start pulse Doppler processing via the input device 13, this function automatically detects a position where the PI value becomes maximum and specifies a desired position P. The control processor 29 automatically sets a sampling position at the specified position P, and executes pulse Doppler processing to acquire, for example, a Doppler waveform like that shown in FIG. 12. Note that the apparatus may automatically determine the execution timing of pulse Doppler processing after automatically setting a sampling position or may determine an execution timing in response to an instruction input by the operator via the input device 13.

### Seventh Modification

The wide-area feature amount image generation function according to the seventh modification displays, at once, feature amounts associated with more areas in the blood vessel to be displayed by concatenating areas in which feature amounts have been measured (feature amount measurement areas).

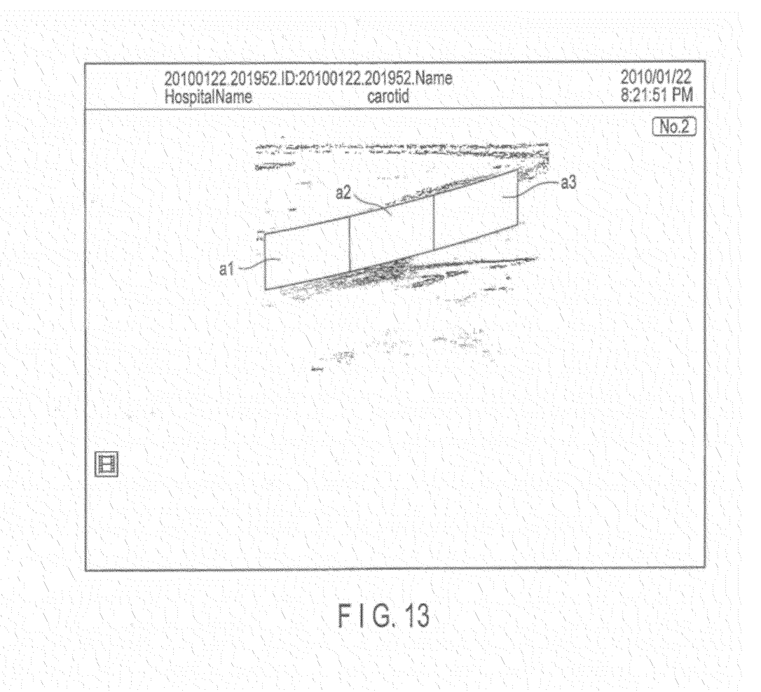

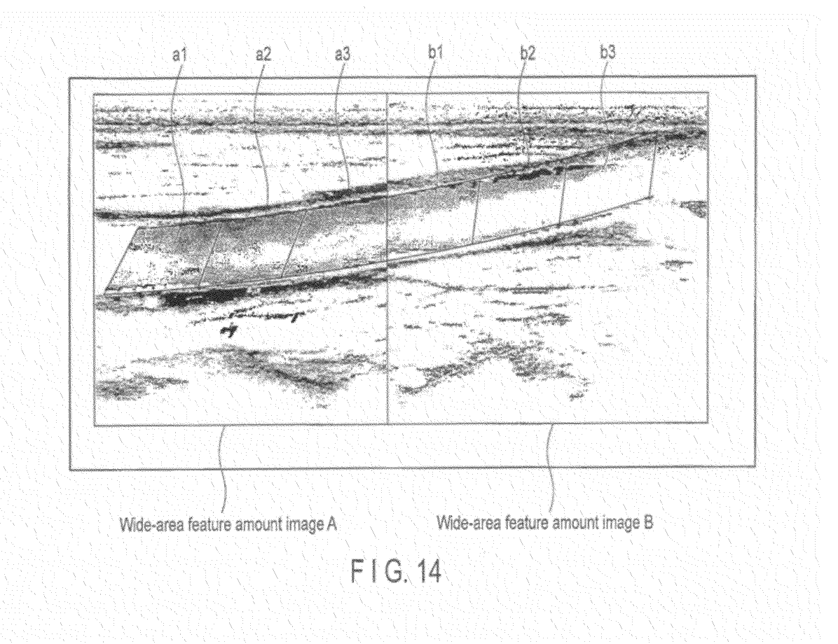

FIGS. 13 and 14 are views for explaining display forms according to the seventh modification. As shown in FIG. 13, concatenating (coupling) feature amount measurement areas a1, a2, and a3 individually calculated in step S3 upon spatially associating them with each other can generate and display a wide-area feature amount image A indicating feature amounts associated with many areas in the blood vessel.

In addition, it is possible to generate and display one composite image like that shown in FIG. 14 by concatenating (coupling) wide-area feature amount images having a plurality of feature amount measurement areas like those shown in FIG. 13 upon spatially associating them with each other in accordance with the method described with reference to the third modification. Such a composite image allows quick and easy visual recognition of blood flow feature amounts in a wider range.

The feature amount calculation in step S3 can be executed by each heartbeat or every plurality of heartbeats. This can be realized by performing the feature amount calculation mentioned in step S3 based on velocity information V (x, y, n) in each frame from the first frame to the nth frame over a heartbeat or a plurality of heartbeats (n is an integer satisfying 1≦n≦N). In the case of generating the concatenated images of FIGS. 13 and 14, the feature amount in each feature amount area is preferred to be calculated by the same number of heartbeats. Further, in the case of obtaining the feature amount by a plurality of heartbeats for each feature amount area, it is also fine to calculate the feature amount for each heartbeat for each feature amount area, calculate the feature amount corresponding to the plurality of heartbeats, and calculate the average thereof.

### Effects

The ultrasonic diagnostic apparatus described above calculates feature amounts such as PI values associated with blood flows at the respective positions in the blood vessel by using blood flow information obtained by CFM over a predetermined interval, and assigns different colors in accordance with the calculated values, thereby generating and displaying a feature amount image. It is therefore possible to calculate feature amounts such as PI values associated with a wide area, as compared with conventional pulse Doppler processing, and to display the result as a wide-area feature amount image. The operator can visually recognize flow velocity index values and feature amounts associated with a wide blood vessel area, as compared with the prior art, quickly and easily by observing the displayed wide-area feature amount image.

The conventional ultrasonic diagnostic apparatus examines the overall blood vessel by moving a sampling position in pulse Doppler processing along the blood vessel, and hence takes much time. In contrast to this, for example, when measuring a blood flow velocity in cervical vessel ultrasonic examination, the apparatus extracts a target blood vessel in a long axis view by using this wide-area feature amount image, and executes screening. This makes it possible to quickly and easily discriminate the presence/absence of an abnormality. This allows the operator to finish examination if there is no abnormality and to perform detailed examination by moving a sampling position in pulse Doppler processing if there is an abnormality, thus improving the examination efficiency.

Note that the present invention is not limited to the embodiment described above, and constituent elements can be modified and embodied in the execution stage within the spirit and scope of the invention.

(1) Each function associated with this embodiment can also be implemented by installing programs for executing the corresponding processing in a computer such as a workstation and expanding them in a memory. In this case, the programs which can cause the computer to execute the corresponding techniques can be distributed by being stored in recording media such as magnetic disks (floppy® disks, hard disks, and the like), optical disks (CD-ROMs, DVDs, and the like), and semiconductor memories.

(2) The blood flow information acquired in the CFM mode may be stored in advance, and this wide-area feature amount image may be generated and displayed afterward.

(3) The above embodiment has exemplified the case in which a wide-area feature amount image is generated and displayed by assigning different colors to corresponding positions in accordance with acquired feature amount values. However, this embodiment is not limited to this case. For example, it is possible to generate and display a wide-area feature amount image by assigning different colors to corresponding positions in accordance with acquired flow velocity index values.

(4) The above embodiment has explained an example in the case of executing wide-area feature amount image generation processing by generating spatial distribution of velocity information using a series of signals obtained by an imaging mode for performing CFM.

However, this embodiment is not limited to this example. It is also possible to execute wide-area feature amount image generation processing by generating spatial distribution of velocity information using a series of signals obtained by other image modes. For example, wide-area feature amount image generation processing may be executed by generating spatial distribution of velocity information using a series of signals obtained by an imaging mode which executes Doppler processing with respect to a series of signals obtained by a B-mode scan. Furthermore, other than the B-mode, spatial distribution of velocity information may be generated by, for example, executing high-speed B-mode scan in which a scanning range is limited, and performing correlation processing (for example, speckle tracking processing) between frames of the obtained B-mode image. The present wide-area feature amount image generation processing is also executable by using such spatial distribution of velocity information.

In addition, various inventions can be formed by proper combinations of a plurality of constituent elements disclosed in the above embodiments. For example, several constituent elements may be omitted from all the constituent elements disclosed in the above embodiments. Furthermore, constituent elements in the different embodiments may be properly combined.

While certain embodiments have been described, these embodiments have been presented by way of example only, and are not intended to limit the scope of the inventions. Indeed, the novel embodiments described herein may be embodied in a variety of other forms;

furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a detection unit configured to detect a distribution of velocity information at each position in a predetermined area in an object over a predetermined interval by scanning the predetermined area with an ultrasonic wave;
a calculation unit configured to calculate at least one feature amount based on at least one of a maximum flow velocity value, a minimum flow velocity value, and a mean flow velocity value at the each position in the predetermined interval by using velocity information at the each position over the predetermined interval; and
a display unit configured to display the feature amount in a predetermined form.

2. The apparatus of claim 1, wherein the feature amount includes at least one of a PI (Pulsatility Index), an RI (Resistance Index), and an S/D.

3. The apparatus of claim 1, wherein the detection unit detects a distribution of velocity information at each position in the predetermined area over a predetermined interval by using a color Doppler mode.

4. The apparatus of claim 1, further comprising an image generation unit configured to generate at least one index image in which different hues are assigned in accordance with respective feature amounts of the at least one feature amount,
wherein the display unit displays the at least one index image.

5. The apparatus of claim 4, wherein the display unit simultaneously displays the at least one index image and a color Doppler image.

6. The apparatus of claim 4, wherein the image generation unit generates a composite image by concatenating the at least one index image upon spatial association, and
the display unit displays the composite image.

7. The apparatus of claim 4, wherein the display unit simultaneously displays the plurality of index images.

8. The apparatus of claim 4, wherein the calculation unit calculates the plurality of feature amounts based on at least one of the maximum flow velocity value, the minimum flow velocity value, and the mean flow velocity value at the each position in the predetermined interval,
the image generation unit generates the index image by using a first feature amount of the plurality of feature amounts, and
the display unit displays, in a predetermined form, the index image generated by using the first feature amount and a second feature amount, of the plurality of feature amounts, which is different from the first feature amount.

9. The apparatus of claim 1, wherein the display unit displays at least one of the flow velocity index value and the feature amount as a graph indicating a spatial change associated with a predetermined path set by an input device.

10. The apparatus of claim 1, further comprising a determination unit configured to determine a sampling position based on at least one of the flow velocity index value and the feature amount when executing a pulse Doppler mode.

11. The apparatus of claim 1, wherein the calculation unit calculates at least one of the flow velocity index value and the feature amount with reference to a heartbeat or pulse.

12. The apparatus of claim 1, wherein the calculation unit calculates the feature amount over a heartbeat or a plurality of heartbeats.

13. The apparatus of claim 1, wherein the detection unit detects the distribution of velocity information based on a series of signals obtained by an imaging mode which executes Doppler processing with respect to a series of signals obtained by a B-mode scan.

14. The apparatus of claim 1, wherein the detection unit detects the distribution of velocity information by executing a speckle tracking process based on a series of signals obtained by a B-mode scan.

15. An ultrasonic image processing apparatus comprising:
a storage unit configured to store velocity information at each position in a predetermined area in an object detected over a predetermined interval by scanning the predetermined area with an ultrasonic wave;
a calculation unit configured to calculate at least one feature amount based on at least one of a maximum flow velocity value, a minimum flow velocity value, and a mean flow velocity value at the each position in the predetermined interval by using velocity information at the each position over the predetermined interval; and
a display unit configured to display the feature amount in a predetermined form.

16. An ultrasonic image processing method comprising:
detecting a distribution of velocity information at each position in a predetermined area in an object over a predetermined interval by scanning the predetermined area with an ultrasonic wave;
calculating at least one feature amount based on at least one of a maximum flow velocity value, a minimum flow velocity value, and a mean flow velocity value at the each position in the predetermined interval by using velocity information at the each position over the predetermined interval; and
displaying the feature amount in a predetermined form.
